(19) United States
(12) Patent Application Publication
(10) Pub. No.: US 2010/0137973 A1
(43) Pub. Date: Jun. 3, 2010

(54) Layered Bifurcation Stent

(75) Inventors: Derek Sutermeister, Eden Prairie, MN (US); James Anderson, Fridley, MN (US); Benjamin Arcand, Minneapoils, MN (US); Jay Rassat, Buffalo, MN (US); Ryan Tschudi, Buffalo, MN (US)

Correspondence Address: VIDAS, ARRETT & STEINKRAUS, P.A., SUITE 400, 6640 SHADY OAK ROAD, EDEN PRAIRIE, MN, 55344, US

(73) Assignee: BOSTON SCIENTIFIC SCIMED, INC., Maple Grove, MN (US)

(21) Appl. No.: 12/615,680

(22) Filed: Nov. 10, 2009

Related U.S. Application Data
(60) Provisional application No. 61/119,138, filed on Dec. 2, 2008.

(51) Int. Cl. A61F 2/82

(52) U.S. Cl. 623/1.15; 623/1.35; 623/1.42

(57) ABSTRACT

An implantable medical device has an outer stent with a tubular body disposed about a longitudinal axis. The outer stent includes members defining openings which are in fluid communication with a flow path. At least one opening is larger than adjacent openings when the outer stent is in an expanded state, and is positioned between the ends of the outer stent. The at least one larger opening is a side branch opening having a perimeter. The device also has an inner stent with a tubular body disposed within the outer stent and about a longitudinal axis. The inner stent has a branch portion with deployable petals positioned adjacent to the side branch opening of the outer stent. The petals are biased against the perimeter of the side branch opening such that the petals are restrained from opening outward when the outer stent is in an unexpanded state.

### CROSS-REFERENCE TO RELATED APPLICATIONS

Not Applicable

### STATEMENT REGARDING FEDERALLY SPONSORED RESEARCH

Not Applicable

### BACKGROUND OF THE INVENTION

1. Field of the Invention

In some embodiments this invention relates to implantable medical devices, their manufacture, and methods of use.

2. Description of the Related Art

A stent is a medical device introduced to a body lumen and is well known in the art. Typically, a stent is implanted in a blood vessel at the site of a stenosis or aneurysm endoluminally, i.e. by so-called “minimally invasive techniques” in which the stent in a radially reduced configuration, optionally restrained in a radially compressed configuration by a sheath and/or catheter, is delivered by a stent delivery system or “introducer” to the site where it is required. The introducer may enter the body from an access location outside the body, such as through the patient's skin, or by a “cut down” technique in which the entry blood vessel is exposed by minor surgical means.

Stents and similar devices such as stent, stent-grafts, expandable frameworks, and similar implantable medical devices, are radially expandable endoprostheses which are typically intravascular implants capable of being implanted transluminally and enlarged radially after being introduced percutaneously. Stents may be implanted in a variety of body lumens or vessels such as within the vascular system, urinary tracts, bile ducts, fallopian tubes, coronary vessels, secondary vessels, etc. They may be self-expanding, expanded by an internal radial force, such as when mounted on a balloon, or a combination of self-expanding and balloon expandable (hybrid expandable).

Stents may be created by methods including cutting or etching a design from a tubular stock, from a flat sheet which is cut or etched and which is subsequently rolled or from one or more interwoven wires or braids.

Within the vasculature it is not uncommon for stenoses to form at a vessel bifurcation. A bifurcation is an area of the vasculature or other portion of the body where a first (or parent) vessel is bifurcated into two or more branch vessels. Where a stenotic lesion or lesions form at such a bifurcation, the lesion(s) can affect only one of the vessels (i.e., either of the branch vessels or the parent vessel) two of the vessels, or all three vessels. Many prior art stents however are not wholly satisfactory for use where the site of desired application of the stent is juxtaposed or extends across a bifurcation in an artery or vein such, for example, as the bifurcation in the mammalian aortic artery into the common iliac arteries.

The art referred to and/or described above is not intended to constitute an admission that any patent, publication or other information referred to herein is “prior art” with respect to this invention. In addition, this section should not be construed to mean that a search has been made or that no other pertinent information as defined in 37 C.F.R. §1.56(a) exists.

All U.S. patents and applications and all other published documents mentioned anywhere in this application are incorporated herein by reference in their entirety.

Without limiting the scope of the invention, a brief summary of some of the claimed embodiments of the invention is set forth below. Additional details of the summarized embodiments of the invention and/or additional embodiments of the invention may be found in the Detailed Description of the Invention below.

A brief abstract of the technical disclosure in the specification is provided for the purposes of complying with 37 C.F.R. §1.72.

### BRIEF SUMMARY OF THE INVENTION

In at least one embodiment, the invention is directed toward an expandable medical device for implantation in a body lumen comprising an outer stent and an inner stent. The outer stent comprises a generally tubular body disposed about a longitudinal axis, and the outer stent has a proximal end, a distal end, and flow path therebetween. The outer stent comprises members which define cell openings, the cell openings being in fluid communication with the flow path. At least one cell opening is larger than adjacent cell openings when the outer stent is in an expanded state. The at least one cell opening is positioned between the proximal end and the distal end of the outer stent. The at least one cell opening is a side branch opening, the side branch opening having a perimeter. The medical device further comprises an inner stent, the inner stent comprising a generally tubular body with a proximal end, a distal end, and a flow path therebetween. The inner stent is disposed within the outer stent and about the longitudinal axis. The inner stent comprises a branch portion positioned adjacent to the side branch opening of the outer stent. The branch portion comprises deployable petals which are biased against the perimeter of the side branch opening such that the petals are restrained from opening outward when the outer stent is in an unexpanded state.

In some embodiments, when the outer stent is in the expanded state, a plurality of petals of the inner stent extends through the side opening of the outer stent.

In at least one embodiment, the inner stent may be a balloon expandable stent or a self-expanding stent, and the outer stent may be a balloon expandable stent or a self-expanding stent.

In some embodiments, the inner stent is engaged to the outer stent, while in some embodiments the inner stent and the outer stent are not engaged to one another.

In at least one embodiment, at least one of the inner stent and outer stent comprises a drug coating.

In some embodiments, the inner stent comprises an inner surface and an outer surface, and the outer stent comprises an inner surface and an outer surface. At least one of surfaces selected from the group consisting of the inner stent inner surface, inner stent outer surface, outer stent inner surface, and outer stent outer surface comprises a drug coating.

In at least one embodiment, the present invention is directed towards a medical device for implantation in a body lumen, the device comprising an outer stent and an inner stent. The outer stent has a generally tubular body with a proximal end and a distal end and is disposed about a longitudinal axis. The outer stent is disposed about the inner stent, with the outer stent and the inner stent in combination defining a flow path. The outer stent comprises members which define cell openings, the cell openings being in fluid communication with the flow path. At least one cell opening has a shape different that that of adjacent cell openings. The at least one cell opening is a side opening and is positioned between the proximal end and the distal end. The inner stent comprises a branch portion positioned adjacent to the side opening of the outer stent. The branch portion comprises deployable petals having a first configuration and a second configuration. In the first configuration the petals are generally flush against the tubular body of the outer stent, and in the second configuration the petals are extended outward with respect to the tubular body of the outer stent through the side opening of the outer stent. At least some of the members of the first stent are constructed and arranged to constrain the branch portion of the second stent from extending when the second stent is in the first configuration.

In some embodiments, the present invention is directed towards an expandable medical device for implantation in a body lumen, the device comprising an outer stent and an inner stent. The outer stent comprises a generally tubular body with a proximal end, a distal end, and a flow path therebetween. The outer stent is disposed about a longitudinal axis. The outer stent comprises members which define cell openings, the cell openings being in fluid communication with the flow path. At least one cell opening is larger than adjacent cell openings when the outer stent is in an expanded state. The at least one cell opening is a side branch opening and is positioned between the proximal end and the distal end. The side branch opening has a perimeter. The medical device further comprises an inner stent having a partially tubular body.

The inner stent is disposed within the outer stent, and the inner stent has a proximal end and a distal end. The inner stent comprises a branch portion positioned adjacent to the side branch opening of the outer stent. The branch portion comprises deployable petals which are biased against the perimeter of the side branch opening such that the petals are restrained from opening outward when the outer stent is in an unexpanded state.

These and other embodiments which characterize the invention are pointed out with particularity in the claims annexed hereto and forming a part hereof. However, for further understanding of the invention, its advantages and objectives obtained by its use, reference should be made to the drawings which form a further part hereof and the accompanying descriptive matter, in which there is illustrated and described embodiments of the invention.

### BRIEF DESCRIPTION OF THE SEVERAL VIEWS OF THE DRAWING(S)

A detailed description of the invention is hereafter described with specific reference being made to the drawings.

FIG. 1A shows a side view of an embodiment of a medical device in an expanded state.

FIG. 1B shows a side view of another embodiment of a medical device in an expanded state.

FIG. 2 shows a top-down view of an embodiment of a stent structure suitable for use as an inner stent in the medical device of FIG. 1.

FIG. 3 shows a top-down view of an embodiment of a stent structure suitable for use as an outer stent in the medical device of FIG. 1.

FIG. 4 shows a top-down view of an embodiment of a medical device comprising the stent structures of FIGS. 1 and 2.

FIG. 5 shows a side view of another embodiment of a medical device with a partially tubular inner stent, in an expanded state.

FIG. 6 shows a cross-sectional view of the embodiment shown in FIG. 5, in an unexpanded state.

FIG. 7 shows a cross-sectional view of an embodiment of a medical device with drug coatings in an unexpanded configuration.

FIG. 8 shows a cross-sectional view of an embodiment of a medical device with drug coatings in an unexpanded configuration.

FIG. 9 shows a cross-sectional view of an embodiment of a medical device with drug coatings in an unexpanded configuration.

FIG. 10A shows a side view of another embodiment of a medical device with an outer stent and an inner stent, in an unexpanded state.

FIG. 10B shows an end view of the embodiment of the medical device depicted in FIG. 10A.

FIG. 11A shows a side view of the embodiment of the medical device depicted in FIG. 10A, in a partially expanded state.

FIG. 11B shows an end view of the embodiment of the medical device depicted in FIG. 11A.

FIG. 12A shows a side view of the embodiment of the medical device depicted in FIG. 10A, in a fully expanded state.

FIG. 12B shows an end view of the embodiment of a medical device depicted in FIG. 12A.

### DETAILED DESCRIPTION OF THE INVENTION

While this invention may be embodied in many different forms, there are described in detail herein specific preferred embodiments of the invention. This description is an exemplification of the principles of the invention and is not intended to limit the invention to the particular embodiments illustrated.

For the purposes of this disclosure, like reference numerals in the figures shall refer to like features unless otherwise indicated.

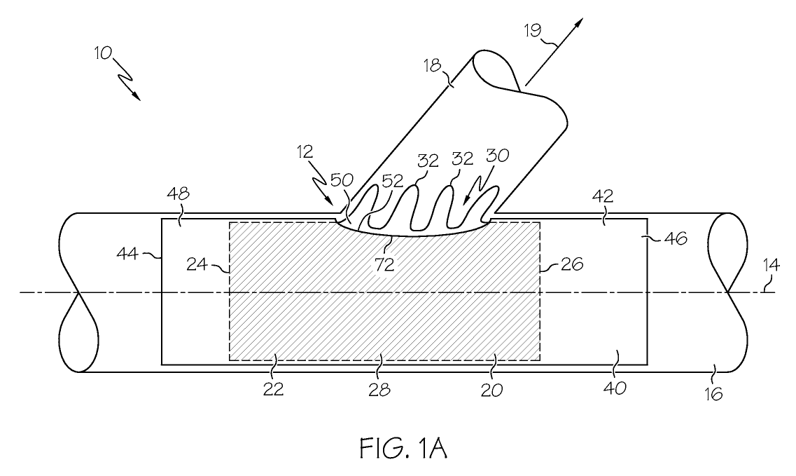

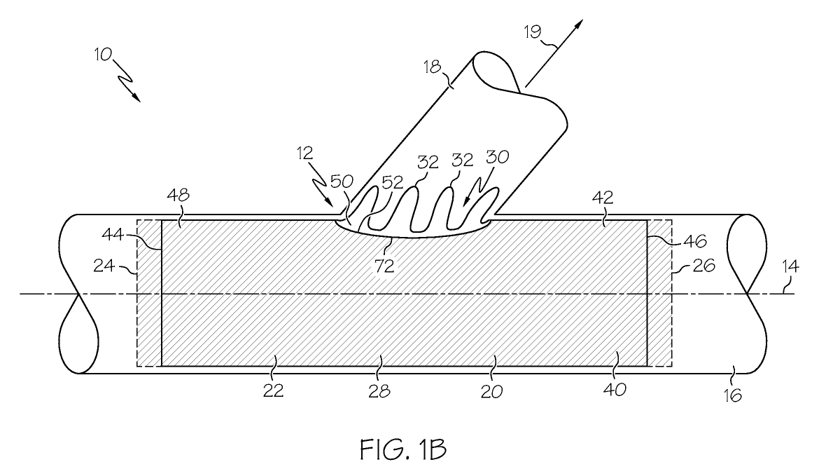

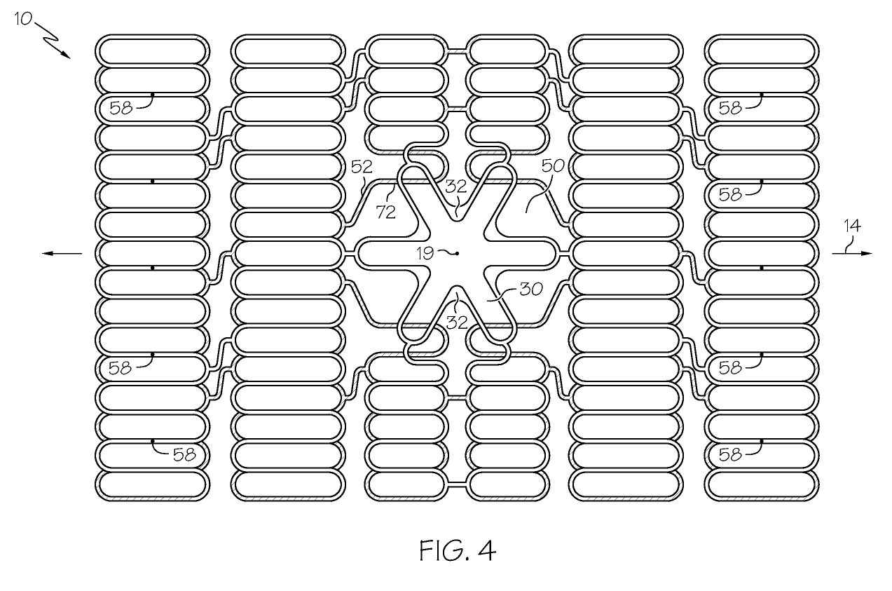

Referring now to FIG. 1A, an embodiment of an expandable medical device 10 is shown in the expanded state at the site of a bifurcation in a body lumen or vessel 12. In one embodiment, the medical device 10 has an inner stent 20, which includes deployable petals 32 constructed from a self-expanding material, and an outer stent 40. In some embodiments, the inner stent is connected to the outer stent at a plurality of connections, as shown in FIG. 4 and described in more detail below.

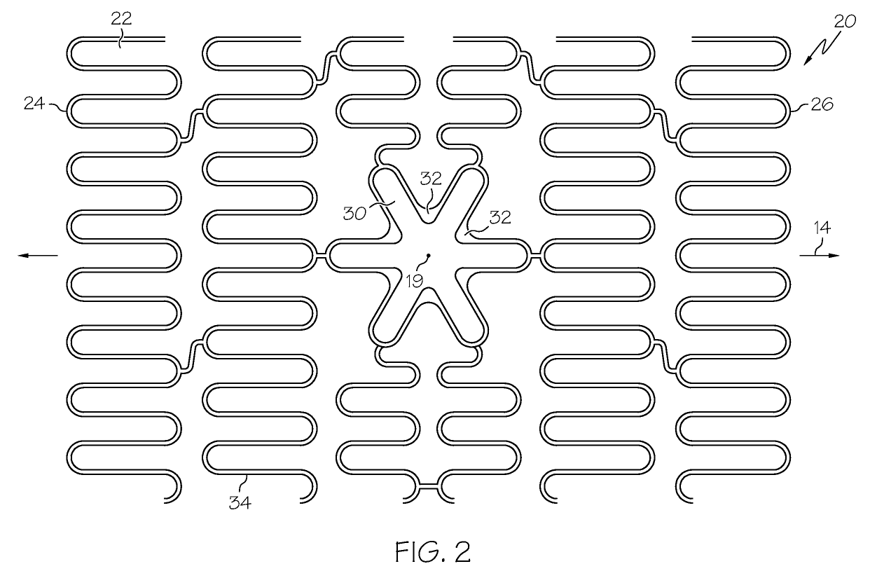

One inventive aspect of the present invention is that in the unexpanded state prior to deployment, portions of the outer stent 40 bias the deployable petals 32 of the inner stent 20 such that the petals 32 are constrained (as shown in FIGS. 2-4). After deployment, the outer stent structure is altered, thereby allowing the deployable petals to extend radially into a branch vessel, like in FIG. 1A. Medical device 10 is disposed about a longitudinal axis 14.

In the embodiment shown in FIG. 1A, the inner stent 20 has a generally tubular stent body 22 disposed about the longitudinal axis 14. Inner stent 20 has a proximal end 24 and a distal end 26, the two ends defining a flow path 28 therebetween.

Still referring to FIG. 1A, the inner stent is shown in an outwardly extended configuration wherein at the site of the bifurcation the branch portion 30 of the inner stent extends from the main body vessel 16 into a side branch vessel 18. Branch portion 30 includes a plurality of deployable petals 32 which are shown in FIG. 1A as being deployed into the side branch 18. Prior to deployment, the petals 32 remain in a configuration that is substantially flush against the inner surface 72 of the outer stent.

Non-limiting examples of overlapping stents may be found in U.S. Patent Application Publication Nos. 2001/0044650 to Simso et al., 2005/0278017 to Gregorich, and 2007/0055362 to Brown et al., the entire contents of each being incorporated herein by reference.

Non-limiting examples of bifurcated stents may be found in U.S. Patent Application Publication Nos. 2005/0119731 to Brucker et al., and 2006/0085061 to Vardi, as well as U.S. Pat. No. 6,168,621 to Vrba, the entire contents of each being incorporated herein by reference.

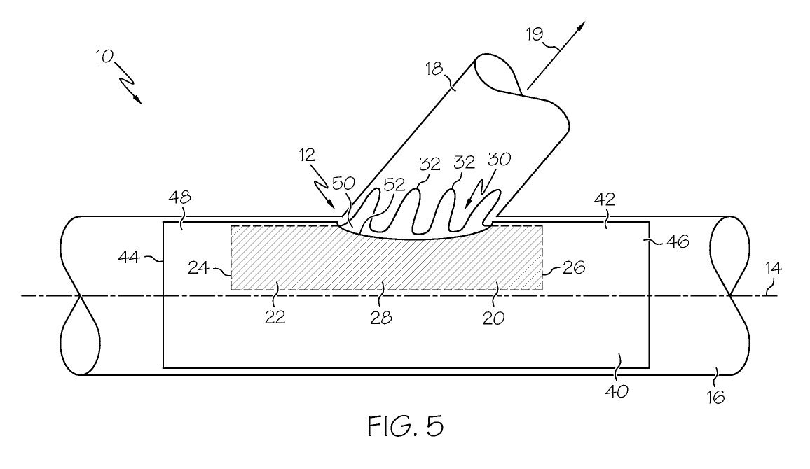

As seen in FIG. 1A, the outer stent 40 is disposed about the inner stent 20. Like the inner stent, outer stent 40 has a generally tubular stent body 42 disposed about the longitudinal axis 14. Outer stent 40 has a proximal end 44 and a distal end 46, the two ends defining a flow path 48 therebetween. The outer stent and the inner stent are substantially coaxially aligned about substantially the same longitudinal axis. As shown in FIG. 1A, because the inner and outer stents are tubular and expand to substantially the same diameter, and because the inner and outer stents lie on substantially the same longitudinal axis, the flow paths 28, 48 of the inner and outer stents are substantially coextensive. In some embodiments, as will be described in more detail below, the inner stent is only partially tubular; the inner stent does not entirely encompass a lumen. That is, in a cross-sectional view of the inner stent, the inner stent does not form a closed path, but instead has a cross-section having an arcuate shape, such as is shown in FIGS. 5 and 6.

The outer stent 40 further includes a side opening 50 being defined by a perimeter 52. FIG. 1A depicts the deployable petals 32 extending outwardly through the side opening 50 and into the side branch vessel 18. In some embodiments, the petals 32 extend outwardly such that the petals are disposed substantially about a longitudinal axis 19 of the side branch vessel.

With the exception of the side branch portion 30, the inner stent of FIG. 1A is shown as being disposed within the tubular body of the outer stent. However, it should be noted that in some embodiments, one or both ends of the inner stent may extend beyond the ends of the outer stent. For example, FIG. 1B depicts both ends (24, 26) of the inner stent 20 extending beyond the ends of the outer stent 40. In the expanded state in such an embodiment, the outer diameter of the inner stent is slightly larger than the outer diameter of the outer stent. As such, a compression fitting is created between the inner stent and the outer stent. Thus, the two stents remain connected after deployment, without the use of connectors, because of the compression fitting.

Turning now to FIG. 2, a flat pattern for a stent that may be suitable for use as the inner stent 20 in the medical device 10 is shown. The members 34 of the inner stent 20 define a side branch portion 30. As mentioned above, the side branch portion 30 comprises deployable petals 32 which when deployed, extend outwardly into the side branch vessel. One of ordinary skill will recognize that petals 32 may have any shape suitable for deployment into a vessel.

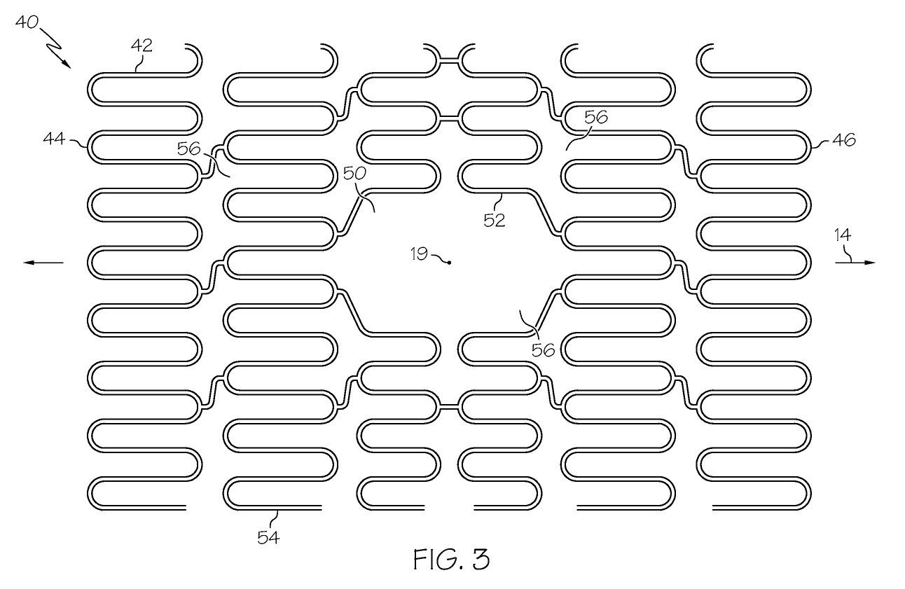

FIG. 3 depicts an embodiment of a flat pattern for a stent that may be suitable for use as the outer stent 40 in the medical device 10. The outer stent 40 is comprised of members 54 which are arranged to define a plurality of cell openings 56.

At least one of the cell openings is larger than any immediately adjacent cell openings. For instance, previously described side opening 50 is a cell opening 56 which is larger than any immediately adjacent cell openings, thereby allowing the petals of the inner stent to deploy into a side branch vessel when the inner and outer stents are properly aligned with one another.

FIG. 4 shows an embodiment of a flat pattern for a medical device 10 comprising the inner stent of FIG. 2 and the outer stent of FIG. 3. A person having ordinary skill in the art will recognize that the flat pattern of FIG. 4 may be rolled to form a cylindrical medical device 10 wherein the outer stent is disposed about the inner stent.

As depicted, the flat pattern for the outer stent is positioned over the inner stent. More specifically, the inner and outer stents are aligned such that the side branch portion 30 of the inner stent is positioned adjacent the side opening 50 of the outer stent. While the medical device 10 is in a first configuration, as shown in FIG. 4, at least a portion of some of the petals 32 of the inner stent are flush against the inner surface 72 of the outer stent. In the embodiment in FIG. 4, the petals 32 are biased against the perimeter 52 of the outer stent's side opening such that the perimeter acts to restrain the petals from opening outwardly into the vessel while the medical device is in an unexpanded configuration. In other words, the outer stent will sheath the inner stent until deployment alters the structure of the cell openings of the outer stent.

In some embodiments, the outer stent is balloon expandable and the inner stent is self-expanding. In such an embodiment, the medical device 10 is delivered to the bifurcation site and the outer stent is expanded using a balloon catheter. As the outer stent is expanded, the side opening also increases in size. As the side opening is increasing in size, the perimeter restrains less of the side branch of the inner stent. Because the inner stent is made from a self-expanding shape memory material, the petals deploy outwardly into the side branch vessel once the perimeter is unable to fully restrain the side branch.

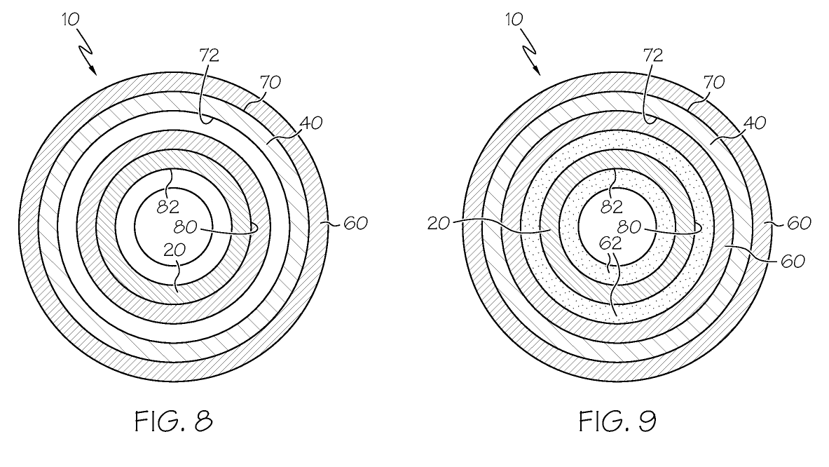

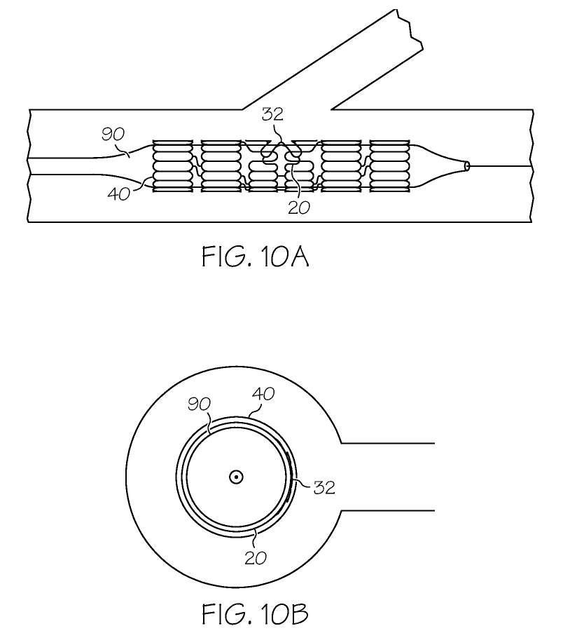

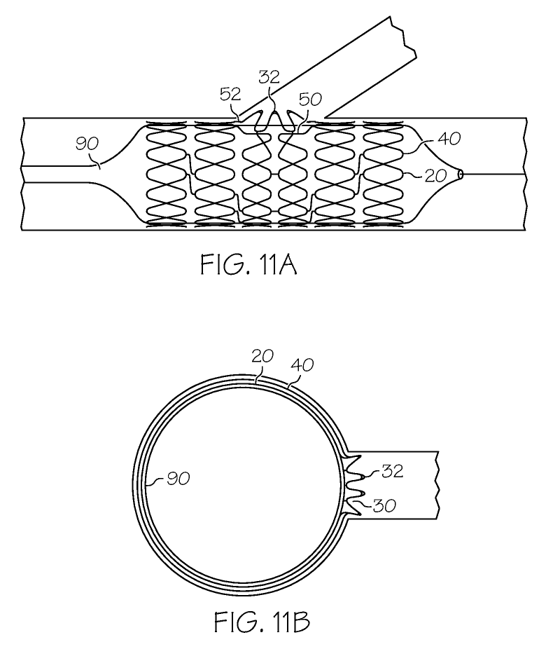

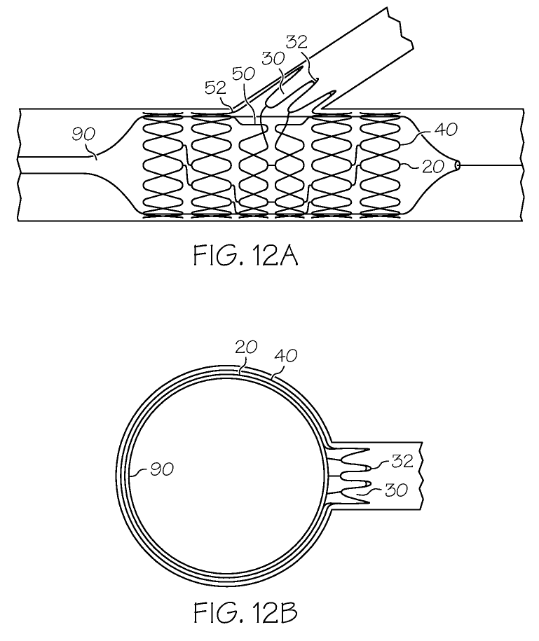

FIGS. 10A-10B depict the balloon expandable outer stent 40 disposed about the self expanding inner stent 20 and the balloon 90 in a body lumen. As seen, both the outer stent 40 and the inner stent 20 are in a non-expanded state. Additionally, self expanding petals 32 are in a non-expanded state; as best seen in FIG. 10B, the petals are constrained by the outer stent 40. FIGS. 11A-11B depict the balloon expandable outer stent 40, the inner stent 20, and the petals 32 in a partially expanded state. As best seen in FIG. 11B, the outer stent 40 has expanded to the point at which petals 32 are no longer fully constrained by the outer stent. FIGS. 12A-12B depict the balloon expandable outer stent 40, the inner stent 20, and the petals 32 in a fully expanded state. The petals 32 are fully deployed into the side branch vessel. The stents in FIGS. 10-12 depict both the outer stent and the inner stent as generally tubular bodies. However, one of ordinary skill in the art will recognize that a partially tubular inner stent would deploy in a manner similar to the inner stent of FIGS. 10-12.

In at least one embodiment, both the outer stent and the inner stent are self-expanding stents. One of ordinary skill in the art will recognize that there are a number of suitable methods of constraining a stent as it is being delivered to a bifurcation site, such as using socks on the ends of the medical device. In an embodiment in which the outer and the inner stent are both made from a shape memory material, the medical device may be configured to allow the outer stent to expand prior to the expansion of the inner stent. For example, if the tubular body of the inner stent is longer than the tubular body of the outer stent, and if socks were used on the ends of the medical device, as the socks were removed, the ends of the outer stent would be released first, thereby allowing it to expand while keeping the inner stent constrained. Once expanded, the socks may be removed completely from the medical device, allowing the inner stent to expand such that its petals deploy through the side opening of the outer stent.

It may also be desirable to configure the medical device such that the outer stent is self-expanding and the inner stent is balloon expandable. In such an embodiment, a sock or other suitable restraint may be removed from the ends of the medical device, thereby allowing it to expand. During or after expansion of the outer stent, a balloon may be used to expand the inner stent, thereby deploying the petals through the side opening of the outer stent.

In some embodiments, as shown in FIG. 4, the inner stent may be connected to the outer stent by at least one and in some embodiments by a plurality of connections 58. A connection 58 may be located on any suitable area of the medical device 10. A connection 58 may comprise any suitable connection between the stents 40, 60. In some embodiments, a connection 58 may comprise a welded, brazed or soldered connection, an adhesive connection, an encapsulated connection, a suture, ring, collar or band, rivets or pins, cooperative tabs and/or notches, friction pads, hook and loop fasteners, etc. In some embodiments, the connections 58 may be insulated, wherein conductivity across a connection 58 is minimized, for example to enhance MRI compatibility.

Referring now to FIG. 5, an embodiment of a medical device 10 is shown. As depicted, the stent body 22 of the inner stent 20 in FIG. 5 is only partially tubular, as compared with the generally tubular stent body of the inner stent in FIG. 1. For example, as shown in the cross-sectional view of FIG. 6, the inner stent 20 extends only partially about the inner circumference of the outer stent 40. Although not depicted, it may be desirable to include connections between the inner and outer stents in the embodiments of FIGS. 5 and 6.

In some embodiments the stent, the delivery system or other portion of the assembly may include one or more areas, bands, coatings, members, etc. that is (are) detectable by imaging modalities such as X-Ray, MRI, ultrasound, etc. In some embodiments at least a portion of the stent and/or adjacent assembly is at least partially radiopaque.

In at least one embodiment, at least a portion of the stent is configured to include one or more mechanisms for the delivery of a therapeutic agent. Often the agent will be in the form of a coating or other layer (or layers) of material placed on a surface region of the stent, which is adapted to be released at the site of the stent's implantation or areas adjacent thereto.

A therapeutic agent may be a drug or other pharmaceutical product such as non-genetic agents, genetic agents, cellular material, etc. Some examples of suitable non-genetic therapeutic agents include but are not limited to: anti-thrombogenic agents such as heparin, heparin derivatives, vascular cell growth promoters, growth factor inhibitors, Paclitaxel, etc. Where an agent includes a genetic therapeutic agent, such a genetic agent may include but is not limited to: DNA, RNA and their respective derivatives and/or components; hedgehog proteins, etc. Where a therapeutic agent includes cellular material, the cellular material may include but is not limited to: cells of human origin and/or non-human origin as well as their respective components and/or derivatives thereof. Where the therapeutic agent includes a polymer agent, the polymer agent may be a polystyrene-polyisobutylene-polystyrene triblock copolymer (SIBS), polyethylene oxide, silicone rubber and/or any other suitable substrate.

Non-limiting examples of medical devices 10 with drug coatings 60, 62 are shown in FIGS. 7-9. In FIG. 7, outer stent 40 has an outer surface 70 and an inner surface 72, each of which is at least partially coated with a drug coating 60. Similarly, inner stent 20 has an outer surface 80 and an inner surface 82, each of which is at least partially coated with drug coating 60.

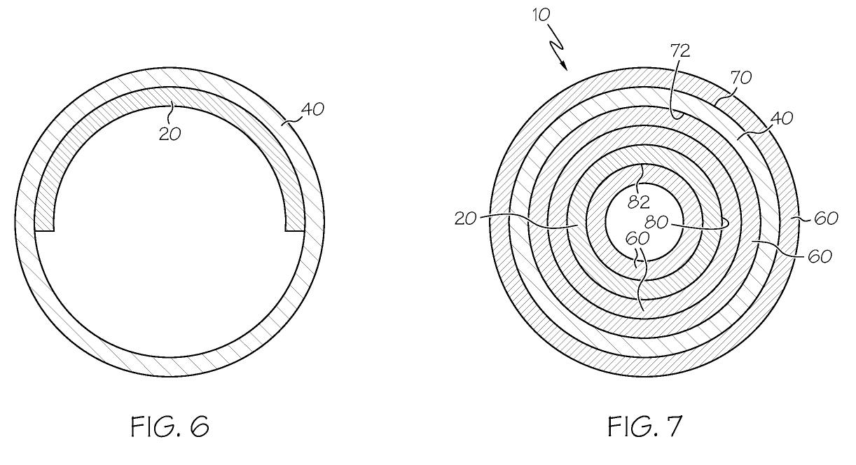

In the embodiment shown in FIG. 8, only the outer surfaces 70, 80 of the outer and inner stents, respectively, include a drug coating 60. It should be noted that in at least one embodiment, it may be desirable to coat the outer stent with a different drug than the inner stent.

Because some vessel bifurcations have more extensive stenotic lesions than adjacent vessel areas, it may be desirable to provide in some embodiments the inner stent with a greater concentration and/or greater quantity of a drug coating or therapeutic agent than that of the outer stent.

In the embodiment shown in FIG. 9, the outer 70 and inner 72 surfaces of the outer stent 40, as well as the outer 80 and inner 82 surfaces of the inner stent 20, are coated with a drug coating, like in FIG. 7. However, in the embodiment in FIG. 9, the outer 70 and inner 72 surfaces of the outer stent 40 are coated with a first drug coating 60, while the outer 80 and inner 82 surfaces of the inner stent 20 are coated with a second drug coating 62.

The above disclosure is intended to be illustrative and not exhaustive. This description will suggest many variations and alternatives to one of ordinary skill in this art. The various elements shown in the individual figures and described above may be combined or modified for combination as desired. All these alternatives and variations are intended to be included within the scope of the claims where the term “comprising” means “including, but not limited to”.

Further, the particular features presented in the dependent claims can be combined with each other in other manners within the scope of the invention such that the invention should be recognized as also specifically directed to other embodiments having any other possible combination of the features of the dependent claims. For instance, for purposes of claim publication, any dependent claim which follows should be taken as alternatively written in a multiple dependent form from all prior claims which possess all antecedents referenced in such dependent claim if such multiple dependent format is an accepted format within the jurisdiction (e.g. each claim depending directly from claim 1 should be alternatively taken as depending from all previous claims). In jurisdictions where multiple dependent claim formats are restricted, the following dependent claims should each be also taken as alternatively written in each singly dependent claim format which creates a dependency from a prior antecedent-possessing claim other than the specific claim listed in such dependent claim below.

This completes the description of the preferred and alternate embodiments of the invention. Those skilled in the art may recognize other equivalents to the specific embodiment described herein which equivalents are intended to be encompassed by the claims attached hereto.

What is claimed is:

1. An expandable medical device for implantation in a body lumen, comprising:
an outer stent, the outer stent comprising a generally tubular body, the outer stent being disposed about a longitudinal axis, the outer stent having a proximal end, a distal end, and flow path therebetween,
the outer stent comprising members, the members defining cell openings, the cell openings in fluid communication with the flow path, at least one cell opening being larger than adjacent cell openings when the outer stent is in an expanded state, the at least one cell opening positioned between the proximal end and the distal end, the at least one cell opening being a side branch opening, the side branch opening having a perimeter; and
an inner stent, the inner stent comprising a generally tubular body, the inner stent being disposed within the outer stent and about the longitudinal axis, the inner stent having a proximal end, a distal end, and a flow path therebetween,
the inner stent comprising a branch portion positioned adjacent to the side branch opening of the outer stent, the branch portion comprising deployable petals, the petals being biased against the perimeter of the side branch opening such that the petals are restrained from opening outward when the outer stent is in an unexpanded state.

2. The medical device of claim 1, wherein when the outer stent is in the expanded state, a plurality of petals of the inner stent extend through the side opening of the outer stent.

3. The medical device of claim 1, wherein the inner stent is selected from the group consisting of balloon expandable stents and self-expanding stents, and wherein the outer stent is selected from the group consisting of balloon expandable stents and self-expanding stents.

4. The medical device of claim 1, wherein the inner stent is engaged to the outer stent.

5. The medical device of claim 1, wherein at least one of the inner stent and outer stent comprises a drug coating.

6. The medical device of claim 5, wherein the inner stent comprises an inner surface and an outer surface, and wherein the outer stent comprises an inner surface and an outer surface, and
wherein at least one of surfaces selected from the group consisting of the inner stent inner surface, inner stent outer surface, outer stent inner surface, and outer stent outer surface comprises a drug coating.

7. An expandable medical device for implantation in a body lumen, comprising:
an outer stent and an inner stent, the outer stent having a generally tubular body and being disposed about a longitudinal axis, the outer stent having a proximal end and a distal end, the outer stent also being disposed about the inner stent, the outer stent and the inner stent in combination defining a flow path, the outer stent and the inner stent in combination having an unexpanded state and an expanded state,
the outer stent comprising members, the members defining cell openings, the cell openings in fluid communication with the flow path, at least one cell opening having a shape different that that of adjacent cell openings, the at least one cell opening positioned between the proximal end and the distal end, the at least one cell being a side opening, the side opening having a perimeter,
the inner stent comprising a branch portion positioned adjacent to the side opening of the outer stent, the branch portion comprising deployable petals, in the unexpanded state the petals being generally flush against at least a portion of the side opening perimeter, in the expanded state the petals extending outward with respect to the tubular body of the outer stent through the side opening of the outer stent.

8. An expandable medical device for implantation in a body lumen, comprising:
an outer stent, the outer stent comprising a generally tubular body, the outer stent being disposed about a longitudinal axis, the outer stent having a proximal end, a distal end, and flow path therebetween,
the outer stent comprising members, the members defining cell openings, the cell openings in fluid communication with the flow path, at least one cell opening being larger than adjacent cell openings when the outer stent is in an expanded state, the at least one cell opening positioned between the proximal end and the distal end, the at least one cell opening being a side branch opening, the side branch opening having a perimeter; and
an inner stent, the inner stent comprising a partially tubular body, the inner stent being disposed within the outer stent, the inner stent having a proximal end, a distal end,
the inner stent comprising a branch portion positioned adjacent to the side branch opening of the outer stent, the branch portion comprising deployable petals, the petals being biased against the perimeter of the side branch opening such that the petals are restrained from opening outward when the outer stent is in an unexpanded state.

9. An expandable medical device for implantation in a body lumen, comprising:
an outer stent and an inner stent, the outer stent having a generally tubular body and being disposed about a longitudinal axis, the outer stent having a proximal end and a distal end, the outer stent also being disposed about the inner stent, the outer stent and the inner stent in combination defining a flow path, the outer stent and the inner stent in combination having an unexpanded state and an expanded state,
the outer stent comprising members, the members defining cell openings, the cell openings in fluid communication with the flow path, at least one cell opening having a shape different that that of adjacent cell openings, the at least one cell opening positioned between the proximal end and the distal end, the at least one cell being a side opening, the side opening having a perimeter,
the inner stent comprising a branch portion positioned adjacent to the side opening of the outer stent, the branch portion comprising deployable petals, in the unexpanded state the petals being generally flush against at least a portion of the side opening perimeter, in the expanded state the petals extending outward with respect to the tubular body of the outer stent through the side opening of the outer stent.
